(19) United States
(12) Patent Application Publication
(10) Pub. No.: US 2020/0397552 A1
(43) Pub. Date: Dec. 24, 2020

(54) BLADDER DEVICE FOR LIGHT BLADDER LEAKAGE

(71) Applicants: James Edward CLAPPERS, Melbourne (AU); Sara CLAPPERS, Melbourne (AU)

(72) Inventors: James Edward CLAPPERS, Melbourne (AU); Sara CLAPPERS, Melbourne (AU)

(21) Appl. No.: 16/883,047

(22) Filed: May 26, 2020

(30) Foreign Application Priority Data
May 24, 2019 (AU) 2019901787

(51) Int. Cl. A61F 2/00

(52) U.S. Cl. CPC A61F 2/0022

(57) ABSTRACT

A device for reducing light bladder leakage having: a. holding member having a resilient deformable shaped body which is sized and resilient to fit within the user's vagina and resiliently return to provide a holding force; b. removing grip attached to the holding member.

### FIELD OF THE INVENTION

The present invention relates to a device for treating light bladder leakage in a human. In particular the present invention relates to a device which is effective for use by sufferers of light bladder leakage urinary incontinence to substantially minimize symptoms of light bladder leakage.

The invention has been developed primarily for use in/with a device for use in treatment of light bladder leakage urinary incontinence to substantially minimize symptoms of light bladder leakage and will be described hereinafter with reference to this application. However, it will be appreciated that the invention is not limited to this particular field of use.

### BACKGROUND OF THE INVENTION

Light bladder leakage also known as urinary stress incontinence is an involuntary loss of urine often during activities that cause pressure on the bladder such as coughing, running, jumping or sneezing.

Light bladder leakage generally occurs a result of weakening of pelvic floor muscles that support the bladder. Weakening of pelvic floor muscles can be caused by pregnancy, previous vaginal births, obesity or chronic urinary tract infections.

Light bladder leakage is a handicap which affects both women and men. According to the Canadian Continence Foundation (2014), as many as 50% of women experience urinary leakage and half again experience light bladder leakage.

A number of devices and methods are known to assist treatment of light bladder leakage. One method involves the use of disposable incontinence pads which usually comprise a shaped pad having a multi-layered core of a super absorbent material. The super absorbent material is capable of absorbing an amount of urine depending on the amount of super absorbent, and retains the urine away from contact with the body to keep skin substantially dry.

Disposable incontinence pads however suffer from a number of drawbacks. For example, once the absorbent core is wet an incontinence pad needs to be changed. It is conceivable that depending on the rate of leakage and type of pad, multiple changes of pad may be required in a single day. A wet incontinence pad may also become heavy, bulky and odorous with urine absorption and therefore uncomfortable, and present an awkward and messy device to dispose of. It is advised that used incontinence pads should not be disposed of by flushing down toilets hence most used incontinence pads are disposed of separately from toileting facilities end up in landfill.

Alternative non-surgical approaches to assist light bladder leakage has included a reusable device which is inserted in the vagina and supporting the urethra to help reduce leaks therefrom. Such a device however has a number of deficiencies including the following:
it can take a few weeks or a couple of months to find the correct placement,
the device can move and fall out during exercising,
an insufficient force is supplied and/or maintained so leaking returns

The present invention seeks to provide an improved device, which will overcome or ameliorate at least one or more of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF INVENTION

According to a first aspect of the present invention, there is provided a bladder device having: a. holding member having a resilient deformable shaped body which is sized and resilience to fit within the user's vagina and resiliently return to provide a holding force; b. removing grip attached to the holding member.

Preferably the device is capable of insertion into the vagina to provide force exertion on adjacent urethra to aid limitation of bladder leakage.

The holding member can be a resilient circumferential shape able to be resilient deformed for insertion into vagina and return to provide resilient pressure against the user's vaginal wall.

The holding member can have a ring structure.

The holding member can include a spaced parallel double ring structure.

The holding member can comprise a spaced parallel double ring structure with membrane between having holes sized to form a partial vacuum when resiliently providing pressure against the user's vaginal wall.

Preferably the removing grip is integral with the holding member.

Preferably the holding member extends substantially in a first plane to provide pressure against opposing walls of the vagina and wherein the removing grip extends substantially in a second plane angled to the first plane and providing a means for the user to grip and remove.

The bladder device can comprise: a. a holding member in the form of a double ring extending in the first plane and forming the resilient deformable shaped body which is sized and resilience to fit within the user's vagina and resiliently return to provide a holding force; b. a first covering extending across the holding member in the first plane; c. a second covering between the double ring having a plurality of openings to provide a vacuum holding effect; d. a removing grip attached to at least part of the first covering or double ring of the holding member; and e. a pathway extending from a first opening on one side of the first covering to a second opening on the other side of the first covering to allow ready removal.

The removing grip can include a body attached to the first covering and or double ring and including a central channel for providing the pathway extending from the first opening on one side of the first covering to the second opening on the other side of the first covering to allow ready removal.

The bladder device preferably comprises: a. a holding member in the form of a double ring extending in the first plane and forming the resilient deformable shaped body which is sized and resilience to fit within the user's vagina and resiliently return to provide a holding force and includes a covering extending between the double ring and having a plurality of openings to provide a vacuum holding effect; b. a removing grip attached to at least part of the double ring of the holding member and forming a first covering extending across the holding member; c. a pathway extending from a first opening in the removing grip on one side of the holding member and extending into an internal channel through to the other side of the holding member.

In a related aspect of the present invention, there is provided a variable geometry device for treating light bladder leakage comprising: a compressible waistband having a series of openings spaced thereabout; a substantially hollow flexible body extending from the waistband; wherein the device is biased to a first geometry by the waistband; wherein the device is inserted within the vagina in a compressed geometry and adopts a second geometry in situ biased towards the first geometry so that the waistband exerts a force on adjacent walls effective to support the urethra; and wherein as the waistband and/or body of the device is compressed, air is expelled through the openings and a vacuum is formed in situ as the device tends towards the first geometry to assist retaining the device in place.

The present invention provides an advance over the prior art as it maintains an effective force on the vaginal walls in situ to support the urethra so as to substantially reduce bladder leakage that might occur during strenuous exercise and occasional pressure from day to day activities and provides improved retention in situ. In addition, the vacuum formed in situ provides an improved retention action so that the device is more able to be retained and withstand stresses associated with strenuous exercising and inadvertent actions such as coughing and sneezing which might otherwise lead to the device being dislodged and/or falling out.

The waistband can comprise at least two spaced apart concentric bands interconnected by a circumferential collar, wherein the collar is recessed relative to the at least two spaced apart concentric bands. The combined spaced apart bands and recessed collar provide an overall structure that is effective for applying sufficient pressure against the vaginal walls to retain the device in location and support for the urethra.

Preferably the series of openings in the waistband are located in the collar. As the waistband and body are compressed for insertion, air exits the openings in the collar, and once in situ the bias expands the body and creates a vacuum that provides a strong means for retention even under stress conditions such as exercising.

Preferably the waistband has a circumference wider than the body. The overall configuration of the device can be substantially conical. The body of the device can comprise a base portion attaching to the waistband, a head portion distal to the waistband and a midsection intermediate the base and head portions. The overall configuration of the body can be substantially conical progressively narrowing towards the head portion.

In one embodiment the head portion can include oppositely disposed openings. The oppositely disposed openings allow easy manual contact and grippability for assisting removal of the device.

Preferably the device is fabricated from a flexible plastic material or rubber or the like which can be compressed for insertion.

In a related aspect of the present invention there is disclosed a method of treating light bladder leakage in a user comprising the method including the steps of: a. providing a variable geometry device having: i. a compressible waistband having a series of openings spaced thereabout; and ii. a substantially hollow body extending from the waistband; iii. wherein the device is biased to a first geometry by the resilience of the waistband; b. squeezing the device so that the waistband folds on itself in an insertion geometry; c, inserting and locating the device within the vagina, waistband first in the insertion geometry; d. releasing the device in situ; e. wherein the device resiliently tends back to the first geometry in situ whereby the waistband unfolds and urges against vaginal walls to aid closing of the adjacent urethra to minimise bladder leakage.

The variable geometry device is inserted within the vagina waistband first in substantially compressed and folded condition. Once located, the waistband unfolds from the insertion geometry in situ towards the first geometry to exert an effective force on vaginal walls to improve support for the urethra.

Furthermore, the step of squeezing the body of the device causes expulsion of air from the body through the openings in the waistband. In this case, when the device is inserted and located in position, and released, the expanding movement of the waistband in situ creates a vacuum which improves retention of the device in situ even in the event of stress including strenuous exercising.

The waistband of the device can comprise at least two spaced apart concentric bands interconnected by a circumferential collar, wherein the collar is recessed relative to the at least two spaced apart concentric bands. The combined spaced apart bands and recessed collar provide an overall structure that provides a resilience such that when released from a folded insertion condition, the device unfolds towards the first geometry so that a substantial force is exerted by the waistband against vaginal walls and thereby to effect support of the urethra.

Preferably the series of openings in the waistband are located in the collar.

Preferably the waistband has a circumference wider than the body. The body of the device can comprise a base portion attaching to the waistband, a head portion distal to the waistband and a midsection intermediate the base and head portions. The overall configuration of the body can be substantially conical progressively narrowing towards the head portion. Preferably the head portion can include oppositely disposed openings. The oppositely disposed openings allow easy manual contact and grippability for assisting removal of the device.

Preferably in the step of squeezing the body, the body is compressed manually by finger force applied thereto and the waistband folded in a u-shape geometry for insertion. Upon release of the body, the resilience of the body and waistband urge unfolding of the waistband from the insertion geometry so the device is moved towards the first geometry and contact of and force applied to the vaginal walls by the waistband.

The device is inserted within the vagina in a compressed geometry and adopts a second geometry in situ biased towards the first geometry so that the waistband exerts a force on adjacent walls effective to support the urethra.

When the waistband and/or body of the device is compressed for insertion, air is expelled through the openings and a vacuum is formed in situ as the device tends towards the first geometry and exerts a force on the walls to retain the device in place.

In a further related aspect of the present invention there is disclosed the use of a variable geometry device for treatment of light bladder leakage, the device comprising a waistband and a body attached to the waistband, wherein the device is biased to a first geometry by the waistband; the device being insertable in the vagina in an insertion geometry and retained in situ in a retention geometry such that the waistband exerts an effective force to vaginal walls to support the urethra.

The waistband can be compressible. The waistband can comprise at least two spaced apart concentric bands interconnected by a circumferential collar, wherein the collar is recessed relative to the at least two spaced apart concentric bands.

The body can be a substantially hollow flexible body extending from the waistband. The body of the device can comprise a base portion attaching to the waistband, a head portion distal to the waistband and a midsection intermediate the base and head portions. Preferably the waistband has a circumference wider than the body. The overall configuration of the body can be substantially conical progressively narrowing towards the head portion.

The head portion of the body can include oppositely disposed openings. The oppositely disposed openings allow easy manual contact and grippability for assisting removal of the device.

In one embodiment of the invention the device can be insertable in an insertion geometry wherein the waistband is configured in a substantially u-shaped geometry wherein at least part of the waistband is folded in on itself. In this geometry the body of the device may be compressed by squeezing. Upon insertion and location, the waistband urges movement of the device towards the first geometry thereby exerting a force on vaginal wall effective for supporting the urethra.

In yet a further related aspect of the present invention there is disclosed a bladder device having: a. a holding member having a resilient deformable shaped body which is sized and with resilience to fit adjacent or within the user's vagina and resiliently provide a peripheral holding force against the urethra; b. a protruding structure connected to the holding member providing a removing grip; wherein the device is capable of insertion to provide force exertion on adjacent urethra to aid limitation of bladder leakage.

Preferably the protruding structure is shaped and connected to the holding member to enable deformation of the resilient deformable shaped body to enable insertion and retraction of the bladder device.

The protruding structure can be frame-like connecting to a peripheral portion of the resilient deformable shaped body. Alternatively the protruding structure can be substantially continuous and connecting to peripheral portion of the resilient deformable shaped body.

Preferably the holding member having a resilient deformable shaped body is shaped to fit within the vagina and the resilient deformable shaped body and/or the protruding structure of the bladder device includes a fluid venting passage.

The present invention represents an advance over prior art systems because it provides improved support of the urethra, it is retained by creation of vacuum in situ hence minimises inadvertent removal or dislodging during stress actions such as exercising, is reusable, comfortable and easily inserted and removed.

Other aspects of the invention are also disclosed with reference to accompanying drawings and examples.

### BRIEF DESCRIPTION OF THE DRAWINGS

Notwithstanding any other forms which may fall within the scope of the present invention, preferred embodiments of the invention will now be described, by way of example only, with reference to the accompanying drawings in which:

FIG. 1 is a schematic representation of a bladder device in accordance with a preferred embodiment of the present invention;

FIG. 2 is a schematic representation in perspective from one side of a device for minimising light bladder leakage in a user in accordance with a preferred embodiment of the present invention;

FIG. 3 is a schematic representation of alternate view of the device of FIG. 1;

FIG. 4 is a schematic vertical cross-section view of the device of FIG. 1 across A-A (as shown in FIG. 5);

FIG. 5 is a schematic representation of the device of FIGS. 1 to 3 shown from a side view;

FIG. 6 is a schematic representation of the device in FIG. 1 in a plan view above the waistband of the present invention;

FIG. 7 is a schematic representation of a device for minimising light bladder leakage in a user in accordance with an alternative preferred embodiment of the present invention;

FIG. 8 is a schematic representation of alternate view of the device of FIG. 6;

FIG. 9 is a photographic representation of a device in accordance with the invention showing variable geometry and method of altering geometry for insertion;

FIG. 10 is a photographic representation of a device in accordance with the invention showing variable geometry and method of altering geometry for insertion;

FIG. 11 is a photographic representation of a device in accordance with the invention showing the configuration of the waistband of the device from a partial top end view ready for insertion in the method of the invention.

FIG. 12 is a schematic representation in a partial perspective bladder device in accordance with a preferred embodiment of the present invention; and

FIG. 13 is a schematic representation in partial perspective of a bladder device in accordance with a further preferred embodiment of the present invention.

### DESCRIPTION OF PREFERRED EMBODIMENTS

It should be noted in the following description that like or the same reference numerals in different embodiments denote the same or similar features.

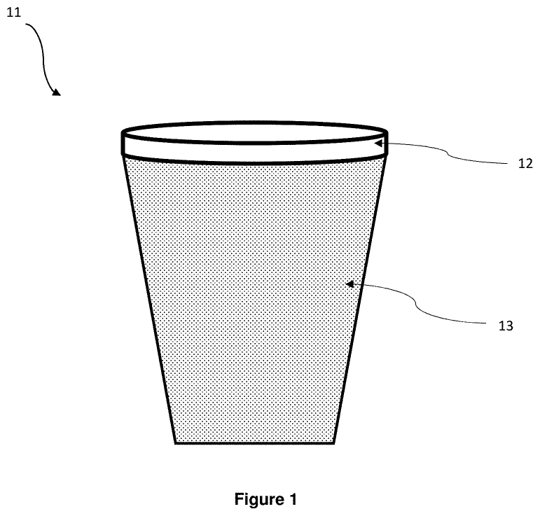

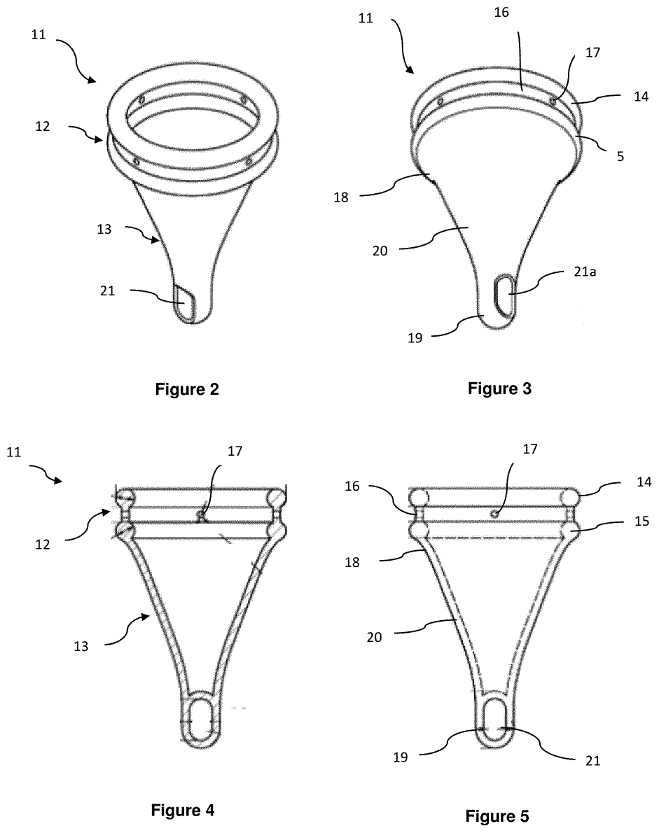

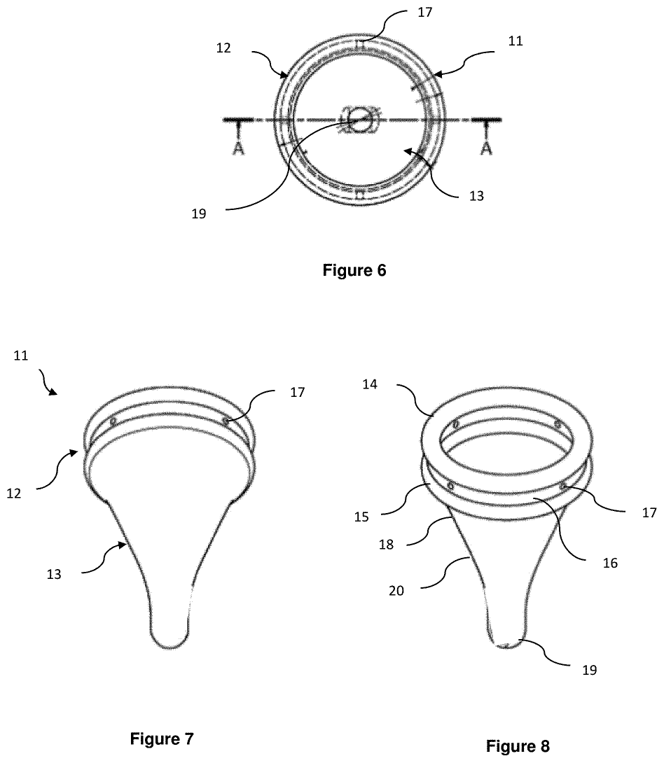

Referring to FIG. 1 there is shown a bladder device 11 including a holding member 12, which can be in the form of a double ring as discussed later (see FIGS. 2 to 11), having a resilient deformable shaped body extending in a first plane, and a removing grip 13 attached to at least part of the holding member extending in a second plane angled to the first plane. The shaped body is sized and resilient to fit within a user's vagina in an insertion condition and resiliently return to provide a holding force.

Preferably the bladder device 11 includes a pathway extending from a first opening in the removing grip (refer FIG. 2) on one side of the holding member and extending into an internal channel through to the other side of the holding member.

As shown in FIGS. 2 to 5, a holding member 12 is illustrated in the form of a double ring 14 and 15 extending in a first plane and forming a resilient deformable shaped body which is sized and resilient to fit within a user's vagina and resiliently return to provide a holding force. The holding member 12 includes a covering or collar 16 extending between the double ring 14 and 15 and having a plurality of openings 21 and 21a to provide a vacuum holding effect. A removing grip 13 is attached to at least part of the double ring of the holding member and forms a covering body extending across the holding member; and a pathway extends from a first opening 21 in the removing grip on one side of the holding member and extending into an internal channel through to the other side of the holding member.

Referring to FIGS. 2 to 10, there is shown a variable geometry device 11 for treating light bladder leakage wherein the holding member is a compressible waistband 12 and the removing grip 13 extending from the waistband 12, whereby the removing grip 13 comprises a substantially hollow body comprising a midsection 20 intermediate base 18 and lower end portion 19.

The variable geometry device 1 as shown in FIGS. 1, 2 and 9 represent an unconstrained bladder device in accordance with the present invention in a first geometry. In use, for the treatment of light bladder leakage, the geometry of the device is altered to adopt an insertion geometry (as shown in FIGS. 10 and 11), and once inserted the waistband 12 urges movement of the device towards the first (unconstrained) geometry (see FIGS. 1, 2 and 9) in which the waistband exerts a force against vaginal walls to thereby support the urethra.

In this embodiment shown in FIGS. 2 to 10, the waistband 12 comprises two spaced apart concentric bands 14 and 15 interconnected by a circumferential collar 16 which is recessed between the spaced apart concentric bands. The spaced apart bands 14 and 15 provide a resilient structure so that the device 11 is biased towards the first unconstrained geometry following insertion in the insertion geometry (shown in FIGS. 10 and 11). Therefore in situ, the structural resilience of the waistband 12 urges movement of the waistband and removing grip 13 away from the insertion geometry (see FIGS. 10 and 11) so that the waistband is effective in applying sufficient force against vaginal walls of the user to retain the device in location and support the urethra for minimising light bladder leakage therefrom.

The collar 16 of the waistband 12 includes a series of openings 17 of pin-hole size spaced thereabout. In an operating condition, when the waistband 12 and/or removing grip 13 of the device is compressed and folded for insertion of the device, air is expelled from the body through the openings 17 in the collar, and a vacuum is formed in situ as the device tends towards the first unconstrained geometry in situ. The vacuum created assists to retain the device in location.

As shown, the removing grip 13 of the device 11 comprises a base portion 18 attaching to the band 15 of the waistband 12, a head or lower end portion 19 distal to the waistband 12 and a midsection 20 intermediate the base and head portion.

The overall configuration of the device 11 shown in the first geometry (FIGS. 1, 3 and 9) is substantially conical, with the waistband 12 forming the widest point, the device progressively narrowing from the waistband towards the head portion 19 of the removal grip. As shown in FIGS. 2 to 5, the head portion 19 of the removal grip 13 can include oppositely disposed openings 21 and 21a therein. The oppositely disposed openings 21 and 21a allow easy manual contact and grippability for assisting removal of the device.

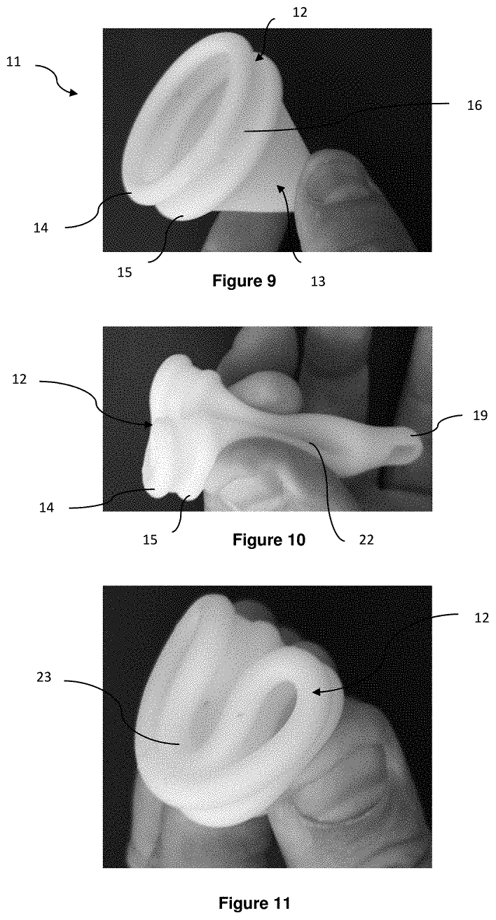

As shown in FIGS. 9 to 11, the device is fabricated from a flexible medical grade plastic material which is adapted to be compressible for insertion in an insertion geometry (see FIGS. 10 and 11) and thereafter resiliently moveable towards the insertion geometry by the resilience of the waistband 12. In this condition the waistband urges against vaginal walls to in turn provide effective support for the urethra. FIG. 9 shows the device in the first geometry in which the device is unconstrained and where the concentric bands 14 and 15 of the waistband 12 bias the device to a substantially conical geometry.

As shown by FIGS. 10 and 11, the variable geometry device 11 is prepared for insertion in an insertion geometry by squeezing the midsection 20 of the removing grip 13. In this condition, the midsection 20 collapses between the finger force forming a fold or crease 22. In so doing, the waistband 12 can be seen folding in on itself (see FIG. 11) in a u-shaped geometry 23 ready for insertion waistband end first. Once located in situ, the resilience of the waistband urges unfolding of the device from the insertion condition towards the first geometry. Consequently, the waistband provides a force against the vaginal walls sufficient to support the urethra and effective to minimise light bladder leakage.

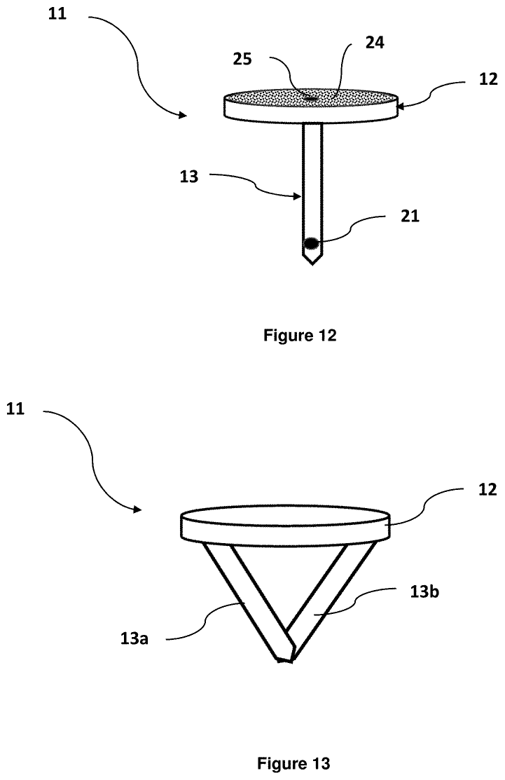

Referring to FIG. 12 a further embodiment of the bladder device is shown in which the bladder device 11 includes a holding member 12 having a resilient body formed by a single ring with a first cover 24 and top opening 25, and a removing grip member 13 which is a substantially elongate and hollow with openings 21 at one end thereof, wherein the top opening 25 and openings 21 form a pathway for airflow therebetween.

The single ring has a resilient deformable shaped body extending in a first plane, and the removing grip 13 attached to at least part of the ring extending in a second plane rectilinear to the first plane. The single ring is sized and resilient to fit within a user's vagina in an insertion condition and resiliently return towards an unconstrained geometry to provide a holding force.

In FIG. 13 there is shown a further embodiment of the bladder device 11 including a holding member 12 having a resilient body formed by a single ring with an open top, and a removing grip formed by elongate hollow members 13a and 13b attached at angles to the holding member.

In all embodiments, there is shown a bladder leakage reduction device comprising a holding structure having a resilient deformable shaped body which is sized and with resilience to fit adjacent or within the user's vagina and resiliently provide a peripheral holding force against the urethra; and a protruding structure connected to the holding structure providing a removing grip. The device is capable of insertion in a deformed configuration and following insertion the device is urged towards a holding configuration by the resilient shaped body. In this way as the device is urged in situ towards the holding condition, the shaped body provides force exertion on or adjacent the urethra to aid limitation of bladder leakage.

As shown, the protruding structure can take numerous forms but is otherwise shaped and connected to the holding structure to enable deformation of the resilient deformable shaped body of the holding structure to enable insertion and retraction of the bladder device. As shown, the protruding structure can be frame-like (see FIG. 13) connecting to a peripheral portion of the resilient deformable shaped body. Alternatively, the protruding structure can be substantially continuous (FIGS. 1 to 11) and connecting to peripheral portion of the resilient deformable shaped body.

As illustrated in the figures, it is preferred that the holding member/structure having a resilient deformable shaped body is shaped to fit within the vagina and the resilient deformable shaped body and/or the protruding structure of the bladder device includes a fluid venting passage between the head portion of the protrusing structure and the resilient deformable shaped body.

Benefits of the system include:
a) the device applies an effective force to continuously support the urethra;
b) the device is comfortable;
c) the device is reusable;
d) the device allows air flow so as to minimise conditions for bacteria propagation and better temperature control;
e) the device provides effective means for substantially preventing inadvertent dislodging or removal during exercising or other triggers such as coughing and sneezing

### Interpretation

### Embodiments

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description of Specific Embodiments are hereby expressly incorporated into this Detailed Description of Specific Embodiments, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Different Instances of Objects

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

Specific Details

In the description provided herein, numerous specific details are set forth. However, it is understood that embodiments of the invention may be practiced without these specific details. In other instances, well-known methods, structures and techniques have not been shown in detail in order not to obscure an understanding of this description.

### Terminology

In describing the preferred embodiment of the invention illustrated in the drawings, specific terminology will be resorted to for the sake of clarity. However, the invention is not intended to be limited to the specific terms so selected, and it is to be understood that each specific term includes all technical equivalents which operate in a similar manner to accomplish a similar technical purpose. Terms such as “forward”, “rearward”, “radially”, “peripherally”, “upwardly”, “downwardly”, and the like are used as words of convenience to provide reference points and are not to be construed as limiting terms.

Comprising and Including

In the claims which follow and in the preceding description of the invention, except where the context requires otherwise due to express language or necessary implication, the word “comprise” or variations such as “comprises” or “comprising” are used in an inclusive sense, i.e. to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means comprising.

Scope of Invention

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

Although the invention has been described with reference to specific examples, it will be appreciated by those skilled in the art that the invention may be embodied in many other forms.

### INDUSTRIAL APPLICABILITY

It is apparent from the above, that the arrangements described are applicable to a bladder device for treating light bladder leakage.

The claims defining the invention are as follows:

1. A bladder device comprising:
a. holding member having a resilient deformable shaped body which is sized and resilience to fit within the user's vagina and resiliently return to provide a holding force; and
b. removing grip attached to the holding member.

2. The bladder device according to claim 1, wherein the device is capable of insertion into the vagina to provide force exertion on adjacent urethra to aid limitation of bladder leakage.

3. The bladder device according to claim 1, wherein the holding member has a resilient circumferential shape able to be resilient deformed for insertion into vagina and return to provide resilient pressure against the user's vaginal wall.

4. The bladder device according to claim 3, wherein the holding member has a spaced parallel double ring structure with membrane between having holes sized to form a partial vacuum when resiliently providing pressure against the user's vaginal wall.

5. The bladder device according to claim 1, wherein holding member extends substantially in a first plane to provide pressure against opposing walls of the vagina and wherein the removing grip extends substantially in a second plane angled to the first plane and providing a means for the user to grip and remove.

6. The bladder device according to claim 1, comprising:
a. holding member in the form of a double ring extending in the first plane and forming the resilient deformable shaped body which is sized and resilience to fit within the user's vagina and resiliently return to provide a holding force;
b. a first covering extending across the holding member in the first plane
c. a second covering between the double ring having a plurality of openings to provide a vacuum holding effect
d. a removing grip attached to at least part of the first covering or double ring of the holding member; and
e. a pathway extending from a first opening on one side of the first covering to a second opening on the other side of the first covering to allow ready removal.

7. The bladder device according to claim 1, wherein the removing grip includes a body attached to the first covering and or double ring and including a central channel for providing the pathway extending from the first opening on one side of the first covering to the second opening on the other side of the first covering to allow ready removal.

8. The bladder device according to claim 1, comprising:
a. holding member in the form of a double ring extending in the first plane and forming the resilient deformable shaped body which is sized and resilience to fit within the user's vagina and resiliently return to provide a holding force and includes a covering extending between the double ring and having a plurality of openings to provide a vacuum holding effect
b. a removing grip attached to at least part of the double ring of the holding member and forming a first covering extending across the holding member; and
c. a pathway extending from a first opening in the removing grip on one side of the holding member and extending into an internal channel through to the other side of the holding member.

9. A variable geometry device for treating light bladder leakage comprising:
a compressible waistband having a series of openings spaced thereabout;
a substantially hollow flexible body extending from the waistband;
wherein the device is biased to a first geometry by the waistband;
wherein the device is inserted within the vagina in a compressed geometry and adopts a second geometry in situ biased towards the first geometry so that the waistband exerts a force on adjacent walls effective to support the urethra; and
wherein as the waistband and/or body of the device is compressed for insertion air is expelled through the openings, and a vacuum is formed in situ as the device tends towards the first geometry to assist retaining the device in place.

10. The variable geometry device according to claim 9, wherein the waistband includes at least two spaced apart concentric bands interconnected by a circumferential collar, wherein the collar is recessed relative to the at least two spaced apart concentric bands.

11. The variable geometry device according to claim 9, wherein the series of openings in the waistband are located in the collar.

12. The variable geometry device according to claim 9, wherein the waistband has a circumference wider than the body.

13. The variable geometry device according to claim 9, wherein the body of the device comprises a base portion attaching to the waistband, a head portion distal to the waistband and a midsection intermediate the base and head portions.

14. The variable geometry device according to claim 9, wherein the head portion includes oppositely disposed openings to allow easy manual contact and grippability for assisting removal of the device.

15. A method of treating light bladder leakage in a user comprising the method comprising the steps of:
a. providing a variable geometry device having:
i. a compressible waistband having a series of openings spaced thereabout; and
ii. a substantially hollow body extending from the waistband;
iii. wherein the device is biased to a first geometry by the resilience of the waistband;
b. squeezing the device so that the waistband folds on itself in an insertion geometry;
c. inserting and locating the device within the vagina, waistband first in the insertion geometry;
d. releasing the device in situ; and
e. wherein the device resiliently tends back to the first geometry in situ whereby the waistband unfolds and urges against vaginal walls to aid closing of the adjacent urethra to minimise bladder leakage.

16. The method of treating light bladder leakage in a user according to claim 15, wherein the step of squeezing the body of the device causes expulsion of air from the body through the openings in the waistband wherein when the device is inserted and located in position, and released, the expanding movement of the waistband in situ creates a vacuum which improves retention of the device in situ even in the event of stress including strenuous exercising.

17. The method of treating light bladder leakage in a user according to claim 15, wherein the waistband of the device comprises at least two spaced apart concentric bands interconnected by a circumferential collar, wherein the collar is recessed relative to the at least two spaced apart concentric bands.

18. A bladder device comprising:
a. a holding member having a resilient deformable shaped body which is sized and with resilience to fit adjacent or within the user's vagina and resiliently provide a peripheral holding force against the urethra;
b. a protruding structure connected to the holding member providing a removing grip, and
wherein the device is capable of insertion to provide force exertion on adjacent urethra to aid limitation of bladder leakage.

19. The bladder device according to claim 18, wherein the protruding structure is shaped and connected to the holding member to enable deformation of the resilient deformable shaped body to enable insertion and retraction of the bladder device.

20. The bladder device according to claim 19, wherein the protruding structure is substantially continuous and connecting to peripheral portion of the resilient deformable shaped body, and wherein the holding member having a resilient deformable shaped body is shaped to fit within the vagina and the resilient deformable shaped body and/or the protruding structure of the bladder device includes a fluid venting passage.
